(19) United States
(12) Patent Application Publication
(10) Pub. No.: US 2004/0248183 A1
(43) Pub. Date: Dec. 9, 2004

(54) Method of identifying and treating insulin-resistant individuals who are responsive to anti-oxidant therapy

(76) Inventors: George Argyropoulos, Baton Rouge, LA (US); Steven R. Smith, Baton Rouge, LA (US)

Correspondence Address: PATENT DEPARTMENT, TAYLOR, PORTER, BROOKS & PHILLIPS, L.L.P, P.O. BOX 2471, BATON ROUGE, LA, 70821-2471, US

(21) Appl. No.: 10/864,622

(22) Filed: Jun. 9, 2004

Related U.S. Application Data
(60) Provisional application No. 60/477,158, filed on Jun. 9, 2003.

(51) Int. Cl. C12Q 1/68

(52) U.S. Cl. 435/6

(57) ABSTRACT

The G/G genotype of a Single Nucleotide Polymorphism (SNP) in the promoter of the human resistin gene, −180C>G, singificantly increased basal promoter activity in adipocytes. These data were recapitulated in vivo where G/G homozygotes had significantly higher resistin mRNA levels in human abdominal subcutaneous fat. A significant interaction was also found between the −180C>G SNP, a marker of oxidative stress and insulin resistance. In addition, resistin mRNA was positively and independently correlated with insulin resistance and hepatic fat as measured by liver X-ray attenuation. These data implicate resistin in the pathophysiology of the human insulin resistance syndrome, an effect mediated by the −180C>G promoter SNP and cellular oxidative stress. The −180C>G SNP may be used to identify insulin-resistant individuals responsive to anti-oxidant therapy, including treatment with anti-oxidant vitamins (e.g. vitamin C, vitamin E, etc.), supplements (e.g., α-lipoic acid, lycopene), foods (e.g., fruits and vegetables) or special diets.

This application claims the benefit of U.S. Provisional Application No. 60/477,158, filed Jun. 9, 2003, under 35 U.S.C. §119(e).

This work was supported in part by a grant from the U.S. Army (DAMD 17-97-2-7013, SRS and GA). The Government has certain rights in this invention.

This invention pertains to a method to identify insulin-resistant individuals (e.g., pre-diabetic, gestational diabetes, increased Cardiovascualar Disease risk, syndrome X, and metabolic syndrome) who will be responsive to anti-oxidant therapy by identifying the presence of a specific allele or by detecting increased resistin levels in blood.

The hormone resistin is a member of a recently-discovered family of cysteine-rich, secreted proteins that are associated with pulmonary inflammation and are expressed in the murine small bowel and adipose tissue. See I. N. Holcomb et al., “FIZZ1, a Novel Cysteine-rich Secreted Protein Associated with Pulmonary Inflammation, Defines a New Gene Family,” Embo. J., vol. 19, pp.4046-4055 (2000). Resistin was shown to be downregulated in the mouse by thiazolidinediones (TZDs), which are agonists for the antidiabetic Peroxisome Proliferator-Activated Receptor γ (PPAR-γ), and which has been proposed to link obesity to diabetes. See C.M. Steppan et al., “The Hormone Resistin Links Obesity to Diabetes,” Nature, vol.409, pp.307-312 (2001). However, the latter findings were contradicted in rodent models of obesity where PPAR-γ agonists augmented expression of resistin. See J. M. Way et al., “Adipose Tissue Resistin Expression Is Severely Suppressed in Obesity and Stimulated by Peroxisome Proliferator-Activated Receptor Gamma Agonists,” J. Biol. Chem., vol. 276, pp. 25651-25653 (2001). At the gene level, certain single nucleotide polymorphisms (SNPS) in non-coding regions of the human resistin gene have been examined and fount not to be significantly associated with insulin resistance, but to be associated with an insulin-sensitivity index. See F. Sentinelli et al., “Human Resistin Gene, Obesity, and Type 2 Diabetes: Mutation Analysis and Population Study,” Diabetes, vol. 51, pp. 860-862 (2002); H. Osawa et al., “Systematic Search for Single Nucleotide Polymorphisms in the Resistin Gene: The Absence of Evidence for the Association of Three Identified Single Nucleotide Polymorphisms with Japanese Type 2 Diabetes,” Diabetes, vol. 51, pp. 863-866 (2002); and H. Wang et al., “Human Resistin Gene: Molecular Scanning and Evaluation of Association with Insulin Sensitivity and Type 2 Diabetes in Caucasians,” J. Clin. Endocrinol. Metab., vol. 87, pp. 2520-2524 (2002).

Resistin expression in humans has been reported at low levels in the adipose tissue of some but not all humans, and its reduced expression has also been proposed to be associated with obesity. See D. B. Savage et al., “Resistin/FIZZ3 Expression in Relation to Obesity and Peroxisome Proliferator-Activated Receptor-Gamma Action in Humans,” Diabetes, vol. 50, pp. 2199-2202 (2001); I. Nagaev et al., “Insulin Resistance and Type 2 Diabetes Are Not Related to Resistin Expression in Human Fat Cells or Skeletal Muscle,” Biochem. Biophys. Res. Commun., vol. 285, pp. 561-564 ( 2001); and S. L. Lay et al., “Decreased Resistin Expression in Mice with Different Sensitivities to a High-Fat Diet,” Biochem. Biophys. Res. Commun., vol. 289, pp. 564-567 (2001).

Resistin mRNA has been reported not to be related to insulin resistance when RNA was isolated from cultured adipocytes, but to be upregulated in acute hyperglycemia in various mouse adipose depots. See J. Janke et al., “Resistin Gene Expression in Human Adipocytes Is Not Related to Insulin Resistance,” Obes. Res., vol. 10, pp. 1-5 (2002); and M. W. Rajala et al., “Cell Type-Specific Expression and Coregulation of Murine Resistin and Resistin-Like Molecule-Alpha in Adipose Tissue,” Mol. Endocrinol., vol. 16, pp. 1920-1930 (2002). Using 32 adipose tissues and quantitative PCR, however, it has been reported that an increased amount of resistin mRNA was present in abdominal depots as compared to thigh depots, suggesting an increased risk for Type 2 diabetes due to central obesity and higher resistin. See C. L. McTernan et al., “Resistin, Central Obesity, and Type 2 Diabetes,” Lancet, vol. 359, pp. 46-47 (2002). The genomic organization and regulation of the murine and human resistin genes are divergent and may explain these discrepant findings. See S. Ghosh et al., “The Genomic Organization of Mouse Resistin Reveals Major Differences from the Human Resistin: Functional Implications,” Gene, vol. 305, pp. 27-34 (2003).

Resistin has also been reported as a cysteine-rich, adipose tissue-specific secretory factor (ADSF) that blocks adipocyte differentiation. See K. H. Kim et al., “A Cysteine-Rich Adipose Tissue-Specific Secretory Factor Inhibits Adipocyte Differentiation,” J. Biol. Chem., vol. 276, pp. 11252-11256 (2001). Failure of adipocyte differentiation has been proposed as a cause of Type 2 diabetes, possibly through an ectopic overload of fatty acids and lipotoxicity of non-adipose tissues. See E. Danforth, Jr., “Failure of Adipocyte Differentiation Causes Type II Diabetes Mellitus?” Nat. Genet., vol. 26, p. 13 (2000); and R. H. Unger et al., “Lipotoxic Diseases of Nonadipose Tissues in Obesity,” Int. J. Obes. Relat. Metab. Disord., vol. 24, suppl 4, pp. S28-32 (2000).

Cellular oxidative stress is the result of normal cellular processes and occurs at the level of the mitochondria when proton potential is high, when free radicals are generated at the plasma membrane, or when cells are exposed to environmental toxins. See S. Raha et al., “Control of Oxygen Free Radical Formation from Mitochondrial Complex I: Roles for Protein Kinase A and Pyruvate Dehydrogenase Kinase,” Free. Radic. Biol. Med., vol. 32, pp. 421-430 (2002); N. Morin et al., “Semicarbazide-Sensitive Amine Oxidase Substrates Stimulate Glucose Transport and Inhibit Lipolysis in Human Adipocytes,” J. Pharmacol. Exp. Ther., vol. 297, pp.563-572 (2001); and P. A. Kern et al., “The Effect of 2,3,7,8-tetrachlorodibenzo-p-dioxin (TCDD) on Oxidative Enzymes in Adipocytes and Liver,” Toxicology, vol. 171, pp. 117-125 (2002). Cellular responses to limit oxidative stress are grouped into Phase I and Phase II enzymes, the latter including heme oxygenase and NAD(P)H:quinone reductase (EC 1.6.99.2). See P. Talalay et al., “Chemoprotection Against Cancer by Phase 2 Enzyme Induction,” Toxicol. Lett., vol. 82-83, pp. 173-179 (1995). NQO-1, a 274-amino-acid flavoprotein, is a prototypical Phase II enzyme that is induced by electrophiles such as tert-butyl hydroxyquinone and oxygen free radicals. See A. K. Jaiswal et al., “Regulation of Genes Encoding NAD(P)H:quinone Oxidoreductases,” Free. Radic. Biol. Med., vol. 29, pp. 254-262 (2000). NQO uses NAD(P)H as an electron donor to reduce and detoxify quinones and their derivatives. NQO-1 knockout mice have an altered intracellular redox state and reduced visceral adipose tissue mass. See A. Gaikwad et al., “In Vivo Role of NAD(P)H:quinone Oxidoreductase 1 (NQO1) in the Regulation of Intracellular Redox State and Accumulation of Abdominal Adipose Tissue,” J. Biol. Chem., vol. 276, pp. 22559-22564 (2001). In view of this growing body of evidence linking insulin resistance and oxidative stress, there may be a role for free radicals in the regulation of adipocyte function. See J. L. Evans et al., “Are Oxidative Stress-activated Signaling Pathways Mediators of Insulin Resistance and Beta-cell Dysfunction?” Diabetes, vol. 52, pp. 1-8 (2003). Oxidative stress impairs insulin action in adipocytes in vitro and in vivo. Treatment with the anti-oxidant lipoic acid improves insulin-stimulated glucose disposal. See A. Rudich et al., “Oxidant Stress Reduces Insulin Responsiveness in 3T3-L1 Adipocytes,” Am. J. Physiol., vol.272, pp. E935-940 (1997); A. Rudich et al., “Lipoic Acid Protects Against Oxidative Stress Induced Impairment in Insulin Stimulation of Protein Kinase B and Glucose Transport in 3T3-L1 Adipocytes,” Diabetologia, vol. 42, pp. 949-957 (1999); A. Tiroshetal., “Oxidative Stress Impairs Insulin but Not Plateletderived Growth Factor Signalling in 3T3-L1 Adipocytes,” Biochem. J., vol. 355, pp. 757-763 (2001); and J. L. Evans et al., “Alpha-lipoic acid: a multifinctional antioxidant that improves insulin sensitivity in patients with type 2 diabetes,” Diabetes Technol. Ther., vol. 2, pp. 401-413 (2000). Studies have demonstrated that oxidative stress helps activate the Sp-1 transcription factor. See R. Ammendola et al., “The DNA-Binding Efficiency of Spl is Affected by Redox Changes,” Eur. J. Biochem., vol. 225, pp. 483-489 (1994); and X.L. Du et al., “Hyperglycemia-induced Mitochondrial Superoxide Overproduction Activates the Hexosamine Pathway and Induces Plasminogen Activator Inhibitor-1 Expression by Increasing SpI Glycosylation,” Proc. Natl. Acad. Sci. USA, vol. 97, pp. 12222-12226 (2000).

A 5′ variant in the human resistin gene, −420C>G, was recently reported to be associated with obesity. See J. C. Engert et al., “5′ Flanking Variants of Resistin Are Associated with Obesity,” Diabetes, vol. 51, pp. 1629-1634 (2002). Significant associations were found of the “G” allele (i.e. the combined C/G and G/G genotypes) with increased BMI, body weight, body fat mass, and waist circumference in men only.

Two publications have reported the existence of a −180C>G SNP in the resistin gene (referred to as −179C>G SNP in some publications in which the numbering scheme counted as nucleotide “−1” the one upstream of the “ATG” translation initiator, instead of counting as nucleotide “−1” the one upstream from the transcription start site which gives −180), but did not report any associations of this SNP with obesity, nor any linkage disequilibrium with other SNPs. See A. Pizzuti et al., “An ATG Repeat in the 3′-Untranslated Region of the Human Resistin Gene Is Associated with a Decreased Risk of Insulin Resistance,” J. Clin. Endocrinol. Metab., vol. 87, pp. 4403-4406 (2002); and X. Ma et al., “Genetic Variants at the Resistin Locus and Risk of Type 2 Diabetes in Caucasians,” J. Clin. Endocrinol. Metab., vol. 87, pp. 4407-4410 (2002).

U.S. patent application No. 2003/0032099 discloses methods for determining an individual's susceptibility to obesity or to a health disorder associated with obesity, comprising detecting an allele at a polymorphic site linked to the resistin gene locus.

U.S. patent application No. 2002/0161210 and International Application No. WO 00/64920 disclose nucleic acids encoding resistin, whose expression is suppressed by antidiabetic compounds; and discloses methods of treating and detecting type 2 diabetes and Syndrome X by modulating resistin expression, or production and activity of the resistin polypeptide.

International Application No. WO 01/96359 discloses a nucleic acid sequence encoding a resistin-like molecule, and methods to treat and diagnose diabetes and other diseases based on the disclosed nucleic acid sequence.

We have discovered a SNP in the proximal promoter of the human resistin gene. We found an association of this SNP with obesity phenotypes in humans. Analysis of homozygous genotypes in cell models showed that this is a functional SNP that affects promoter activity, and therefore affects gene expression in vivo. Resistin mRNA was measured in subcutaneous fat in humans and was found to have a statistically significant correlation to the −180 SNP. We also showed significant interactions between the −180 SNP, resistin mRNA, and oxidative stress in vivo using NQO mRNA as a measure of oxidative stress. The results indicated that the −180 SNP could be used to identify insulin-resistant individuals who would be responsive to anti-oxidant therapy. Individuals with the G genotype of this SNP had high resistin levels only when there was also evidence of oxidative stress. This SNP may be used to identify insulin-resistant individuals (e.g., pre-diabetic, gestational diabetes, increased Cardiovascular Disease risk, Syndrome X, metabolic syndrome) who would respond to therapy that reduces cellular oxidative stress. Such therapies, for example, include treatment with anti-oxidant vitamins (e.g. vitamin C, vitamin E, etc.), supplements (e.g., a lipoic acid, lycopene), foods (e.g., fruits and vegetables), or special diets.

### BRIEF DESCRIPTION OF DRAWINGS

FIG. 1 illustrates the structure of the human resistin gene (top), the relative positions ofthe putative binding sites for transcription factors (middle), and the position of the −180C>G SNP and electropherograms of the two homozygous genotypes (bottom).

FIG. 2 illustrates luciferase activity measured in cells transiently co-transfected with various promoter contructs and control vectors, expressed as Relative Luciferase Activity (RLA) calculated as a fold-increase over the activity of the pGL3-basic vector.

FIG. 3 illustrates the LS means from an ANOVA analysis ofthe relationship between NQO expression (a measure of oxidative stress), genotype, and resistin mRNA.

FIG. 4 illustrates the LS means from an ANOVA analysis of the relationship between HOMA-IR (a measure of insulin resistance), genotype, and NQO expression (a measure of oxidative stress).

FIG. 5a illustrates the LS means from an ANOVA analysis of the relationship between insulin resistance (as measured by HOMA-IR) and resistin mRNA.

FIG. 5b illustrates the LS means from an ANOVA analysis of the relationship between the amount of liver fat (as measured by the liver/spleen ratio) and resistin MRNA.

### EXAMPLE 1

Research Design and Methods

Promoter of Resistin

The gene structure and promoter region for resistin/FIZZ3 was determined from sequences in the Bacterial Artificial Chromosome (BAC) with GenBank Accession number AC008763, sequences for the C/EBPε regulated myeloid-specific secreted cysteine-rich protein precursor gene (HXCP1) with Accession number AF352730, and BLAST analyses ofthe cDNA with Accession number NM_{—}020415. Nucleotide +3168 on the sequence with accession number AF352730 was designated as the putative transcription start site (nucleotide number, “−1”).

Sequence Alignments and Algorithmic Analyses

Sequence alignments between the mouse and human resistin promoter or thologous regions were performed with a Web-based algorithm (http://saturn.med.nyu.edu/searching/prsa.cgi; Skirball Institute of Biomolecular Medicine, New York University School of Medicine, New York, N.Y.). The human DNA sequence used was from Genbank accession number AF352730, and the mouse sequence was taken from H. B. Hartman et al., “Mechanisms Regulating Adipocyte Expression of Resistin,” J. Biol. Chem., vol. 277, pp. 19754-61 (2002). Algorithmic analyses to identify predicted recognition binding sites for transcription factors in the promoter of resistin were performed with the following software: ‘TESS: Transcription Element Search Software on the WWW’, Jonathan Schug and G. Christian Overton, Technical Report CBIL-TR-1997-1001-v0.0, of the Computational Biology and Informatics Laboratory, School of Medicine, University of Pennsylvania, 1997, URL: http://www.cbil.upenn.edu/tess.

The −180C>G Promoter Polymorphism

The −180C>G Single Nucleotide Polymorphism (SNP) in the human resistin promoter was identified in the SNP database (Sanger Center, Cambridge, England) with dbSNP accession number 273660. The positional assignment for this SNP was based on the assignment of “+1” to the transcription start site at nucleotide 3168 in the sequence with Accession number AF352730. Genotyping of individuals for the −180C>G SNP was performed on a LI-COR DNA Analyzer 4200 (Lincoln, Nebr.). Amplicons were generated with the primers FZ3P1: 5′-TTTTGTCATGTTTGCATCAGC-3′ (SEQ ID NO: 1) and FZ3P2: 5′-GGGCTCAGCTAACCAAAT-3′ (SEQ ID NO: 2). PCR conditions were as follows: one cycle at 95° C. for 4 minutes, followed by 30 cycles, each consisting of denaturation step at 95° C. for 1 minute, an annealing step at 60° C. for 1 minute, and an extension step at 72° C for 1 minute. The sequencing reactions were performed as prescribed by the manufacturer (LI-COR, Lincoln, Nebr.) with the following primer tagged with the 795 nm IRDye 800: 5′-GACCAGTCTCTGGACATGAA-3′ (SEQ ID NO: 3).

Transfection Reporter Constructs

Reporter constructs were generated by amplification of genomic DNA from two homozygous individuals (C/C & G/G), and cloning of the PCR amplicons into the pGL3-basic luciferase reporter vector (Promega, Madison, Wis.). The PCR primers were the FZ3P1 and FZ3P2 primers described above with the addition of leader sequences that corresponded to the Sac I and Xho I restriction enzymes to facilitate directional cloning into the pGL3-basic vector. PCR products and a pGL3-basic vector plasmid preparation were treated with these two enzymes as prescribed by the manufacturer (New England Biolabs, Beverly, Mass.), purified from agarose gel slices, and incubated into a ligation reaction at 16° C. overnight in the presence of T4 DNA ligase (New England Biolabs, Beverly, Mass.). Following transformation of competent DH5α cells (GIBCO-BRL, Gaithersburg, Md.), transformants were grown in LB/Amp media, and preparations were made for the plasmids representing the two genotypes: −303/+27(C/C) and −303/+27(G/G). The genotypes of the two constructs were confirmed by bi-directional sequencing of the plasmids performed by Perkin Elmer (Wellesley, Mass.).

Cell Culture and Luciferase Assay

3T3-L1 cells were differentiated by incubation for 48 hours in the following reagents: DMEM (Cellgro, Fisher, Pittsburgh, Pa.) supplemented with 10% fetal bovine serum (FBS), 100 units/ml penicillin, 100μg/ml streptomycin, 830 nM insulin, and 2 μM dexamethasone. Subsequently, the dexamethasone was removed from the media and the cells were maintained in the differentiated state in the presence of insulin for an additional 10 days. The cells were initially cultured in plastic dishes (10 cm diameter), at 37° C. in a humidified atmosphere with 5% CO₂. For transfections, differentiated 3T3 L1 cells (grown an additional 10 days after removal of dexamethasone) were grown to 85% confluence in six-well (34.8 mm well diameter) plates (Corning, Corning, N.Y.). Cells were transiently co-transfected with the constructs and β-gal plasmids for 24 hours in the absence of serum using Geneporter2 transfection reagent (Gene Therapy Systems, San Diego, Calif.), as otherwise described in D. K. Mayfield et al., “A Role for the Agouti Related Protein Promoter in Obesity and Type 2 Diabetes,” Biochem. Biophys. Res. Commun., vol. 287, pp. 568-573 (2001). According to the kit manufacturer (Gene Therapy Systems, San Diego, Calif.), the transfection efficiencies of the 3T3 L1 cells were estimated at 30-40%, using a β-gal reporter plasmid. The media were then supplemented with 20% fetal calf serum (FCS) for 24 hours. Cells were harvested using 1x Geneporter2 lysis buffer, and the lysates were assayed for luciferase 9 and β-galactosidase activities, as prescribed by the assay manufacturer (Promega, Madison, Wis.) in a luminometer (Zylux Corporation, Pforzheim, Germany). The pGL3-basic vector was used as a negative control, and the pGL3-promoter vector was used as a positive control. Luciferase activity measurements were normalized to β-galactosidase values. Luciferase activity was also measured for untransfected cells. For each cell line, each transfection was carried out in duplicate and the experiments were repeated a minimum of five times for each construct. The mean Luciferase Relative Activity (LRA) for each construct was calculated as a fold-increase over that for the negative control vector (pGL3-basic). The data presented represent the means of five duplicate, independent, transfection experiments per construct.

Human Studies

The Pennington Biomedical Research Center Institutional Review Board approved these studies, and all participants provided written informed consent prior to participation. Vital signs, laboratory values, body fat, and visceral adipose tissue mass were measured as otherwise described in S. R. Smith et al., “Contributions of Total Body Fat, Abdominal Subcutaneous Adipose Tissue Compartments, and Visceral Adipose Tissue to the Metabolic Complications of Obesity,” Metabolism, vol. 50, pp. 425-435 (2001).

Association Study (Cohort 1)

The population consisted of 978 individuals who enrolled in a variety of clinical research studies at the Pennington Biomedical Research Center over the prior 5 years. Each participant signed an informed consent. Volunteers were excluded for a diagnosis of diabetes as defined by a fasting plasma glucose≧126 mg/dL. Blood glucose and insulin values were available for 671 of these individuals. Body composition was determined by DEXA and visceral adipose tissue (cm²) measured by single slice CT scanning as described above.

Measurement of Resistin mRNA in Human Adipose Tissue (Cohort 2)

Cohort 2 included 58 otherwise healthy but overweight and obese men and women. Each participant signed an informed consent. Body composition was determined by DEXA and visceral adipose tissue (kg) measured by multi-slice CT scanning as described above. Liver fat (X-ray attenuation value—Houndfield Units) was measured by CT scanning with splenic X-ray attenuation as an intrascan control. A low liver-spleen ratio is indicative of a fatty liver, whereas a high liver-spleen ratio is indicative of low fat in the liver. See L. Ryysy et al., “Hepatic Fat Content and Insulin Action on Free Fatty Acids and Glucose Metabolism Rather than Insulin Absorption Are Associated with Insulin Requirements During Insulin Therapy in Type 2 Diabetic Patients,” Diabetes, vol. 49, pp. 749-758 (2000). HOMA-IR was calculated as the product of fasting insulin (μU/ml) and glucose values (mmol) divided by 22.5. See D. R. Matthews et al., “Homeostasis Model Assessment: Insulin Resistance and Beta-cell Function from Fasting Plasma Glucose and Insulin Concentrations in Man,” Diabetologia, vol. 28, pp. 412-419 (1985).

After local anesthesia, adipose tissue was collected by needle biopsy from the superficial subcutaneous adipose tissue lateral to the umbilicus, and was then frozen. Total RNA was isolated from approximately 50 mg of frozen tissue using Trizol (Life Technologies, Gaithersburg, Md.). RNA was dissolved in Formazol and stored at −80° C. The concentration and quality of the RNA was determined spectrophotometrically. The TaqMan Real Time RT-PCR technique (Applied Biosystems, Foster City, Calif.) was used to quantitatively measure levels of human resistin mRNA. The primer-probe set for human resistin was designed using Primer Express software (Perkin Elmer, Wellesley, Mass.) as follows: forward primer 5′-AGCCATCAATGAGAGGATCCA-3′ (SEQ ID NO: 4), reverse primer 5′-TCCAGGCCAATGCTGCTTA-3′ (SEQ ID NO: 5), and the “molecular beacon” (5′ FAM-TCGCCGGCTCCCTAATATTTAGGGCA-bhq-1-3′ (SEQ ID NO: 6); Biosearch Technologies Inc., Novato, Calif.) that spans the exon2/exon3 boundary. The reverse transcription and polymerase chain reaction (RT-PCR) were carried out in one tube using an ABI PRISM 7700 SDS instrument (Applied Biosystems, Foster City, Calif.) with 10 ng total RNA as template. The master mix included 300 nM primers, 100 nM probe, reverse transcriptase MuLV, RNase inhibitor, and AmpliTaq GOLD™ (Applied Biosystems, Foster City, Calif.). The Ct value for every sample was measured in duplicate, and resistin mRNA levels were normalized to cyclophilin mRNA levels (Applied Biosystems, Foster City, Calif., Cat #: 4310883E).

Measurement of HQO mRNA (Cohort 2)

The same procedures as described above for resistin RNA were followed for the measurement of NQO mRNA using the following primers: forward primer 5′-TCATTCTCTGGCCAATTCAGAGT-3′ (SEQ ID NO: 7), reverse primer 5′-GGAGTGTGCCCAATGCTATATG-3′ (SEQ ID NO: 8), and probe 5′ FAM-TCTGTGGCTTCCAAGTCTTAGAACCTCAACTG-BHQ1-3′ (SEQ ID NO: 9) (Biosearch Technologies Inc., Novato, Calif.).

Statistical Analyses

Genotype relationships with clinical and laboratory variables were determined using one-way ANOVA, two-way ANOVA, or mixed models (JMP v 4.0, SAS Institute, Cary, N.C.). Multivariate regression was used to test the significance of the relationship of resistin mRNA to HOMA-IR, liver fat, body fat, visceral fat, and gender in the models. Sample means are presented unless otherwise noted, however, variables were log-normalized prior to analysis where necessary. The results of the general linear model are graphically presented as leverage plots that illustrate the residuals of the independent variable after adjusting for the other variables in the model. Analyses were repeated for Cohort 2 using ethnicity (race) and gender as covariates, or using Caucasians only (also adjusted for gender). The data could not be analyzed for the African Americans alone because of their low numbers in Cohort 2 (CC=0, GC=11, GG=1).

### EXAMPLE 2

Identification of Single Nucleotide Polymorphism, −180C>G, in the Resistin Promoter

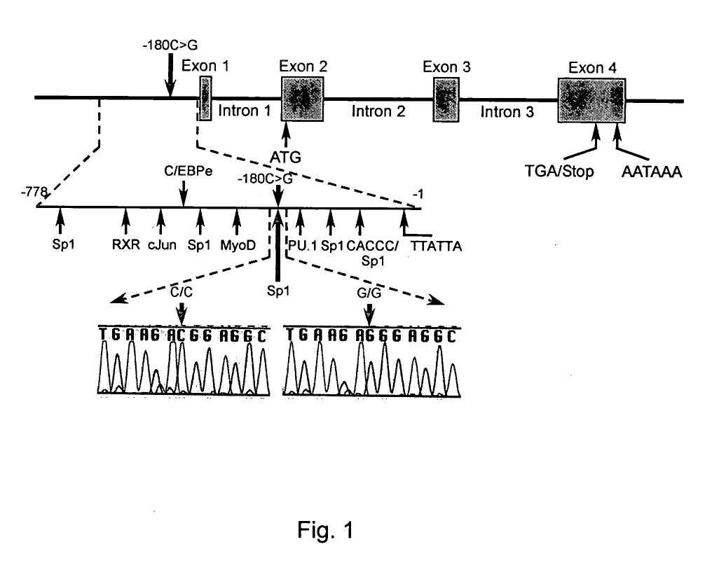

The gene structure of the human resistin coding sequence and the minimal promoter ofthe gene were determined from known genomic and cDNA sequences. FIG. 1 shows the structure of the human resistin gene. Resistin comprises three coding exons and a 5′ non-coding exon (top panel). The relative positions of the putative binding sites for transcription factors are indicated in the middle panel. The position of the −180C>G SNP and electropherograms (bottom panel) of the two homozygous genotypes are shown. Several putative binding sites for transcription factors, and a non-canonical TATA-box (TTATTA), were identified by algorithmic analyses, and included a recognition domain for the transcription activators of the C/EBP family that is commonly found in fat cells. See H. Weintraub et al., “MyoD Binds Cooperatively to Two Sites in a Target Enhancer Sequence: Occupancy of Two Sites Is Required for Activation,” Proc. Natl. Acad. Sci. USA, vol. 87, pp. 5623-5627 (1990). Several putative recognition binding sites for the Sp1/Sp3 family of transcription factors were found at high frequencies.

A search of the human Single Nucleotide Polymorphism database (dbSNP) was performed. A polymorphism was identified 180 nt upstream of the putative transcription start site, where a cytosine was substituted by a guanine (−180C>G). The −180C/C genotype is represented in FIG. 1 by the seventh nucleotide (“C”) in the thirteen nucleotide sequence of TGAAGACGGAGGC (SEQ ID NO: 10). Correspondingly, the −180G/G genotype is represented in FIG. 1 by the seventh nucleotide (“G”) in the thirteen nucleotide sequence of TGAAGAGGGAGGC (SEQ ID NO: 11). This substitution resulted in the gain or loss of predicted recognition binding sites for several transcription factors, including the multifaceted Specific protein Sp1, X2 box binding protein, MalT, R1-R2, TCF-1, TFII-I, and CAC-binding protein. Alignments of the proximal promoters of the human and mouse resistin orthologs showed an overall conservation of the sequences at the 57.6% level, with higher conservation (88%) of the sequences flanking the TTATTA-box, but lower conservation (50%) of the sequences flanking the −180C>G SNP region. The SNP itself was not conserved between the two species.

### EXAMPLE 3

Functional Properties of the −180C>G SNP

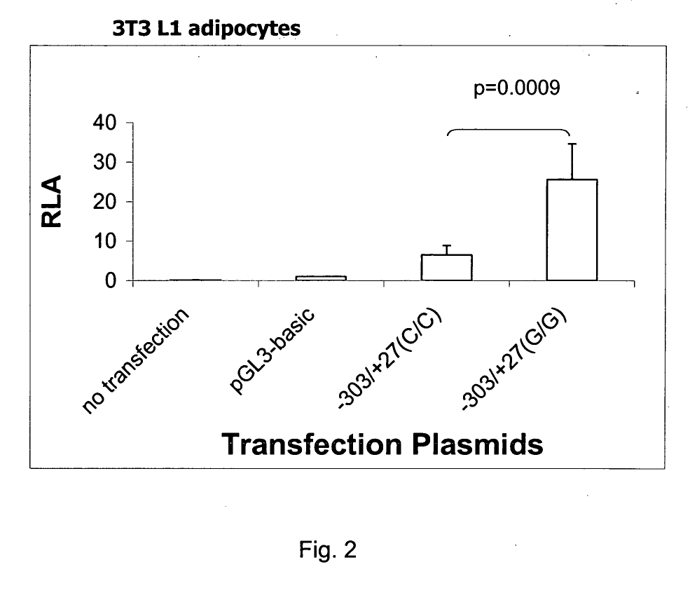

The impact of the −180C>G SNP on promoter activity of the corresponding region was assessed through two identical constructs (330 nt long) in the pGL3-basic vector that differed only in the corresponding polymorphism, −303/+27(C/C) and −303/+27(G/G). The luciferase reporter gene was used to measure promoter activity. Differentiated 3T3 L1 cells were transiently co-transfected with the promoter constructs or control vectors. Luciferase values were averaged, and Relative Luciferase Activity (RLA) was calculated as a fold-increase over the activity of the pGL3-basic vector. FIG. 2 shows luciferase activity as measured in transiently transfected 3T3 L1 cells. The data represent the means of a minimum of five duplicate experiments per transfection construct. RLA denotes the Relative Luciferase Activity, compared to that of the control pGL3-basic vector. Statistical significance of difference between the constructs representing the two genotypes (C/C & G/G) was calculated by Student's t-test. The −303/+27(G/G) construct had a four-fold higher luciferase activity than the −303/+27(C/C) construct in the differentiated 3T3 L1 adipocytes (FIG. 2).

A functional SNP, −180C>G, was then identified in the promoter of the resistin gene. The polymorphic region had significant promoter activity, which parallels the findings reported here for the or thologous mouse region, where the proximal 264 nt of the mouse resistin promoter sufficed for expression of the gene in adipocytes, possibly through binding of the adipogenic transcription factor C/EBPα. Transfection of differentiated 3T3L1 cells with the two polymorphic constructs showed that the G/G genotype had four-fold higher promoter activity than the C/C genotype.

### EXAMPLE 4

The −180C>G SNP and Human Body Fat Levels

To explore the effects of the −180C>G SNP on human physiology, 978 individuals (Cohort 1—Table 1) with known body composition phenotypes (% fat and visceral adipose tissue by CT scanning) were genotyped. Genotype frequencies (CC=49.8; CG=40.9; GG=9.3) were in Hardy-Weinberg equilibrium. The G/G genotype of the −180C>G SNP was significantly associated with higher BMI and higher body fat percent in Caucasian men, but not in women, or in African Americans of either gender (Table 1). However, after performing Bonferroni corrections for multiple testing this association was found not to be significant (Table 1). No significant effects for the −180C>G SNP were found on fasting insulin values, with or without adjusting for any effects of gender, body fat, and visceral fat.

The possible association of the −180C>G SNP with obesity/diabetes phenotypes was also examined in Cohort 1. G/G homozygous males (i.e., individuals with the high promoter activity genotype) were significantly more obese, as measured by BMI and percent body fat, than were allozygotes (includes both G/C heterozygotes and C/C homozygotes), but the significance was eliminated after adjusting for ethnicity and for multiple comparisons by the Bonferroni method.

TABLE 1
(Cohort 1) Population characteristics according to the three −180 C. > G
genotypes (N = sample size, F = female, M = male, AA = African
American, C = Caucasian, NS = not significant, VAT = Visceral
Abdominal Tissue measured at L4-5). Values are sample means ± standard
error of the mean, except for body fat and visceral fat, which are presented
as least square means ± standard error of the mean. P-values are
by one-way ANOVA, except where noted.

| | C/C | C/G | G/G | P-value |
|---|---|---|---|---|
| N (frequency | 487 (49.8%) | 400 (40.9%) | 91 (9.3%) | |
| %)* | | | | |
| Gender | 299/188 | 272/128 | 59/32 | |
| (M/F) | | | | |
| Ethnicity | 79/408 | 74/326 | 18/73 | |
| (AA/C) | | | | |
| Age | 48.5 + 0.48 | 49.41 + 0.49 | 47.91 + 1.02 | NS |
| BMI (kg/m2) | | | | |
| M | 28.9 + 0.24 | 28.8 + 0.26 | 29.3 + 0.62 | NS§ |
| F | 29.1 ± 0.34 | 28.6 ± 0.35 | 28.5 ± 0.82 | NS |
| Fatness (%) | | | | |
| M | 29.1 + 0.54 | 30.2 + 0.66 | 32.3 + 1.33 | NS¶ |
| F | 44.8 + 0.43 | 44.0 + 0.45 | 41.9 + 0.97 | NS |
| VAT (cm2)‡ | 120.0 ± 3.14 | 114.5 ± 3.45 | 109.7 ± 6.27 | NS |
| Glucose | 5.5 + 0.02 | 5.46 + 0.03 | 5.37 + 0.06 | NS |
| (mmol) | | | | |
| Insulin | 76.8 + 2.9 | 85.4 + 3.3 | 63.8 + 7.1 | NS |
| (pmol/L) | | | | |

*: Genotype frequencies were in Hardy-Weinberg equilibrium
§: P = 0.07 by ANOVA and P = 0.03 between GG & CC by post-hoc analysis, in Caucasian men only
¶: P = 0.017 by ANOVA and P = 0.025 between GG & CC by post-hoc analysis, in Caucasian men only
‡: VAT was adjusted for total body fat and gender

### EXAMPLE 5

Resistin Expression in Human Adipose Tissue

A different Cohort, consisting of 58 unrelated individuals (Cohort 2), was used to measure resistin expression using total RNA isolated from subcutaneous fat biopsies and real time RT-PCR. (See Table 2) Resistin expression in gluteal femoral and subcutaneous abdominal depots was highly correlated (R2=0.59, P=0.0001; data not shown). Genotype frequencies were in Hardy-Weinberg equilibrium (Table 2), but differed from the frequencies in Cohort 1, probably due to the small sample size and a recruitment bias in the sample for overweight and hyperinsulinemic individuals (who tended to be G/G homozygotes—Table 2). Data were adjusted for ethnicity and gender.

There was no relationship between resistin mRNA and age, body fatness, visceral adiposity, BMI, a serum marker of inflammation (C-reactive protein, data not shown) or adipose tissue TNF-alpha mRNA expression (data not shown). However, there was a genotype effect. G/G homozygotes had significantly higher resistin mRNA than allozygotes both before (data not shown) and after adjusting the statistical model for ethnicity and gender (Table 2). When performing the same analysis for Caucasians only, G/G homozygotes had significantly higher resistin expression than did allozygotes (Table 2). After adjusting for multiple testing (10 tests) the P-value remained significant for the ethnicity-adjusted dataset (P=0.02) and for the Caucasians only (P=0.02). The same analysis could not be performed for African Americans because of the low sample number (Table 2).

TABLE 2
(Cohort 2) population characteristics according to the three −180 C. > G
genotypes (N = sample size, F = female, M = male, AA-African
American, C = Caucasian, NS = not significant, VAT = Visceral
Abdominal Tissue measured by multi-slice CT scanning, AU = arbitrary
units). Analyses were adjusted for ethnicity and gender. Values are sample
means ± standard error of the mean. The P-values are by ANOVA.

| | C/C | C/G | G/G | P-value |
|---|---|---|---|---|
| N (frequency %)* | 19 (33%) | 29 (50%) | 10 (17%) | |
| Gender (F/M) | 3/16 | 9/20 | 4/6 | |
| Ethnicity (AA/C) | 0/19 | 10/19 | 1/9 | |
| Age | 43.9 ± 2.5 | 41.5 ± 2.1 | 39.6 ± 3.4 | NS |
| BMI (kg/m2) | 32.1 ± 0.9 | 32.5 ± 0.7 | 32.2 ± 1.8 | NS |
| Fatness (%) | 42.7 ± 1.8 | 39.2 ± 1.3 | 41.2 ± 2.6 | NS |
| Insulin (pmol/L) | 96.9 ± 13.5 | 95.4 ± 9.6 | 106.2 ± 19.8 | NS |
| Glucose (mmol) | 5.5 ± 0.2 | 5.6 ± 0.1 | 5.8 ± 0.3 | NS |
| HOMA-IR (AU) | 3.2 ± 0.6 | 3.3 ± 0.4 | 3.7 ± 0.8 | NS |
| Liver/spleen (AU) | 1.29 ± 0.1 | 1.20 ± 0.07 | 1.14 ± 0.14 | NS |
| VAT (kg)‡ | 3.8 ± 0.5 | 3.7 ± 0.3 | 3.9 ± 0.6 | NS |
| NQO mRNA | 0.30 ± 0.04 | 0.30 ± 0.03 | 0.36 ± 0.06 | NS |
| (AU) | | | | |
| Resistin | 1.01 ± 1.1 | 1.97 ± 0.8 | 6.3 ± 1.4_{§} | 0.002 |
| mRNA (AU) | | | | |
| Resistin | 0.75 ± 1.03 | 1.50 ± 1.0 | 5.9 ± 1.3_{§} | 0.002 |
| mRNA (AU)# | | | | |

*: Genotype frequencies were in Hardy-Weinberg equilibrium
‡: VAT was adjusted for fatness, ethnicity, and gender
#: Caucasians only, adjusted for gender
_{§}: significantly different from C/C and C/G genotypes

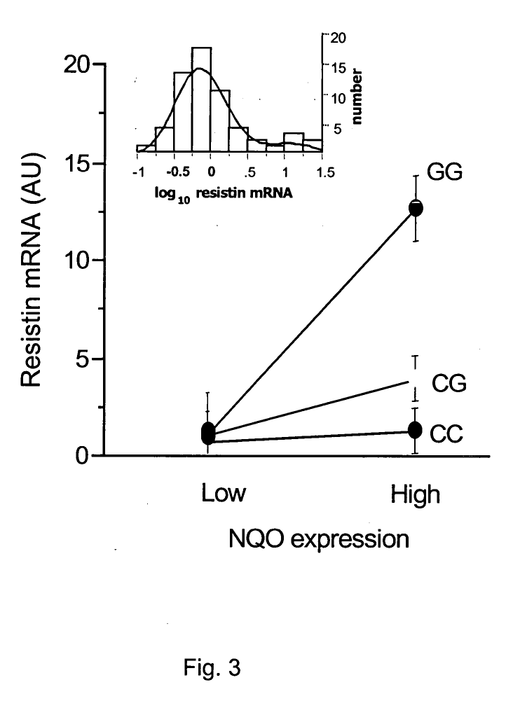

For several individuals with high resistin mRNA levels, resistin mRNA was measured in a second adipose tissue sample taken eight weeks after the first. Resistin MRNA levels were similar in the two samples (data not shown). I.e., resistin mRNA expression was stable over time over the course of the experiment. High expression in G/G homozygotes was not merely transient. However, although all C/C homozygotes had low resistin MRNA, not all G/G homozygotes exhibited high resistin mRNA. There was a bimodal distribution of resistin mRNA levels in Cohort 2 (FIG. 3, inset). It appeared that there was an additional, then-unknown factor involved in the regulation of resistin other than the −180C>G genotype.

### EXAMPLE 6

Interactions Between the −180C>G SNP, Oxidative Stress, and Resistin

We conducted experiments to test the hypothesis that oxidative stress might interact with the G/G genotype to increase resistin mRNA. NQO mRNA (a measure of oxidative stress) was measured in subcutaneous fat of Cohort 2. We found that genotype and NQO mRNA were both related to resistin expression.

Oxidative stress was measured by the expression levels of NQO, a typical Phase II antioxidant enzyme. NQO knockout mice have been reported to have an altered intracellular redox state, and to have reduced visceral adipose tissue mass. See A. Gaikwad et al., “In Vivo Role of NAD(P)H:quinone Oxidoreductase 1 (NQO1) in the Regulation of Intracellular Redox State and Accumulation of Abdominal Adipose Tissue,” J. Biol. Chem., vol. 276, pp. 22559-22564 (2001).

Cohort 2 was divided into high and low expressors of NQO mRNA (i.e. upper and lower 50th percentiles). NQO mRNA was not significantly different between the three −180 SNP genotypes (Table 2). Using mRNA as the dependent variable, significant effects of both the genotype and oxidative stress were observed (Genotype, P=0.005; NQO, P=0.0008; [NQO×Genotype], P=0.08 by ANOVA). After adjusting for ethnicity and gender, a similar result was observed (Genotype, P=0.006; NQO, P=0.0007; [NQO×Genotype], P=0.08) See FIG. 3. However, when Caucasians only (adjusted for gender) were tested, there was a significant interaction between Genotype, NQO mRNA, and Resistin MRNA (Genotype, P=0.01, NQO, P=0.003, [NQO×Genotype], P=0.05).

There was no direct correlation between the −180C>G SNP and NQO mRNA. The causes of high oxidative stress in human fat cells could result from many factors including high fat diet, inactivity, smoking, etc. Analyses of the data showed a significant interaction between the −180C>G SNP, NQO MRNA, and Resistin MRNA, but this interaction did not hold significance after adjusting for ethnicity and gender, which could be due to the disproportionate frequencies of the genotype in the African Americans of this Cohort. This possibility was confirmed when examining the Caucasians only (adjusted for gender), where the interaction between Genotype, Resistin MRNA, and NQO MRNA was significant.

### EXAMPLE 7

Interactions Between the −180C>GSNP, Oxidative Stress, and Insulin Resistance

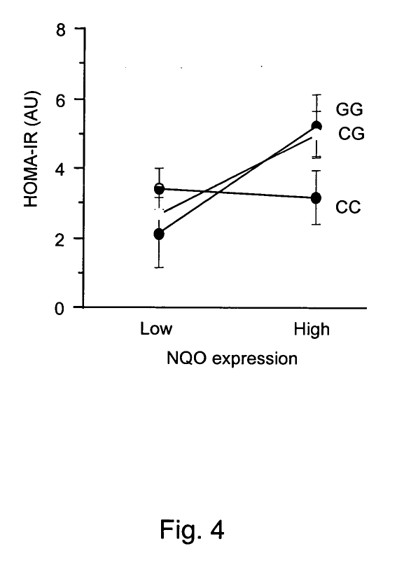

We tested our hypothesis that the SNP and oxidative stress interacted with respect to insulin resistance (measured by HOMA-IR). There was a significant relationship between the −180 SNP, oxidative stress, and HOMA-IR (Genotype, P=0.6; NQO, P=0.004; [NQO×Genotype], P=0.03). (FIG. 4) Because there were two ethnic groups and genders in the dataset (Cohort 2), data were subsequently adjusted using ethnicity and gender as covariates. The interactions between Genotype, NQO mRNA, and HOMA-IR remained significant (Genotype, P=0.72; NQO, P=0.004; [Genotype×NQO], P=0.03). The same analysis was performed for Caucasians only (adjusted for gender) with essentially the same results (Genotype, P=0.46; NQO, P=0.005, [NQO×Genotype], P=0.04).

There was a significant interaction illustrating an effect of the SNP alone and oxidative stress, but only for the GG genotype. These data suggest that the resistin −180C>G SNP and cellular oxidative stress are related to insulin resistance syndrome in humans.

### EXAMPLE 8

Resistin mRNA is Related to Insulin Resistance and Hepatic Fat Accumulation

To test our hypothesis that high resistin MRNA (irrespective of genotype) was related to insulin resistance syndrome and ectopic fat storage, we constructed multivariate models with insulin resistance (as measured by HOMA-IR) and the amount of liver fat (as measured by the liver/spleen ratio) as dependent variables in Cohort 2.

Fatty liver is also a component of insulin resistance syndrome, and contributes to increased hepatic glucose output. See L. Ryysy et al., “Hepatic Fat Content and Insulin Action on Free Fatty Acids and Glucose Metabolism Rather than Insulin Absorption Are Associated with Insulin Requirements During Insulin Therapy in Type 2 Diabetic Patients,” Diabetes, vol. 49, pp. 749-758 (2000).

We found resistin mRNA levels to be related to insulin resistance and to ectopic fat deposition. Data are presented as LS means from ANOVA. After adjusting for the effects of body fat, visceral adiposity, ethnicity, and gender, resistin mRNA in abdominal subcutaneous tissue was proportionally and positively correlated with insulin resistance as measured by HOMA-IR (P=0.003) or when performing the analysis in Caucasians only adjusted for gender (P=0.01).

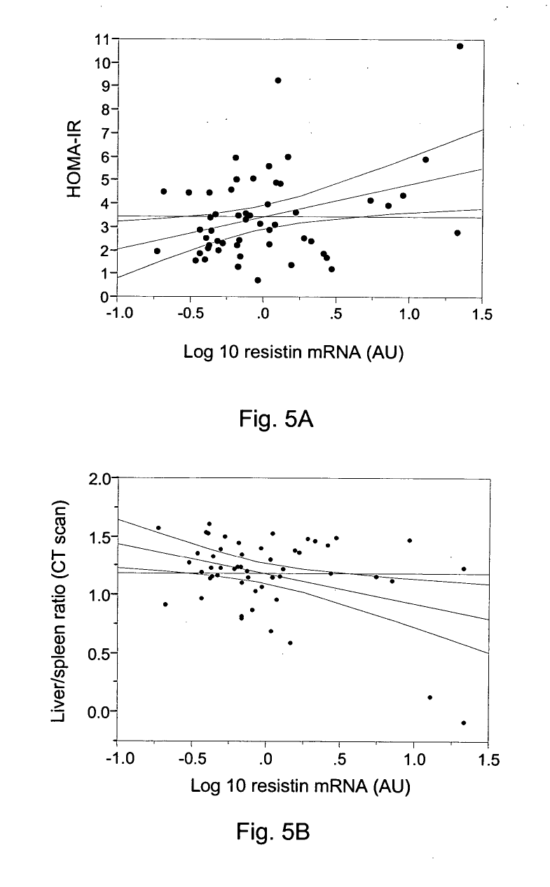

High resistin expression was proportionally related to insulin resistance (P=0.003) and ectopic/hepatic fat (P=0.008), after adjusting for the effects of body fat and visceral adiposity. See FIG. 5a. This effect remained after adjusting for ethnicity and gender in the case of HOMA-IR, (P=0.02; FIG. 5a) and liver fat, (P=0.008; FIG. 5b) The same analysis was also performed in Caucasians only, adjusted for gender, and the effect remained significant for both measures (HOMA-IR, P=0.01; liver fat, P=0.003).

Our results showed that resistin mRNA is directly and independently related to liver fat infiltration.

The G/G genotype of a Single Nucleotide Polymorphism (SNP) in the promoter of the human Resistin gene, −180C>G, significantly increased basal promoter activity in adipocytes. These data were recapitulated in vivo where G/G homozygotes had significantly higher resistin mRNA levels in human abdominal subcutaneous fat. A significant interaction was also found between the −180C>G SNP, a marker of oxidative stress [NAD(P)H quinone oxidoreductase mRNA; NQO], and insulin resistance (HOMA-IR). In addition, resistin MRNA was positively and independently correlated with insulin resistance and hepatic fat as measured by liver X-ray attenuation. These data implicate resistin in the pathophysiology of human insulin resistance syndrome, an effect mediated by the −180C>G promoter SNP and cellular oxidative stress.

The term “therapeutically effective amount” as used herein refers to an amount of an anti-oxidant compound sufficient to decrease insulin resistance in an individual to a statistically significant degree (p<0.05). Examples of anti-oxidant compounds include anti-oxidant vitamins (e.g., vitamin C, vitamin E, vitamin A, vitamin B1, Vitamin B6, Vitamin B12, riboflavin, etc.), supplements (e.g., α-lipoic acid, lycopene, selenium, niacin, folic acid, biotin, pantothenic acid, choline bitartrate, inositol, PABA, lutein, zeaxanthin, Coenzyme Q10, etc.), foods (e.g., fruits and vegetables), or special diets. The term “therapeutically effective amount” therefore includes, for example, an amount sufficient to prevent the occurrence of an insulin-resistant disease. Generally, the dosage will vary with the age, weight, and condition of the individual. A person of ordinary skill in the art, given the teachings of the present specification, may readily determine suitable dosage ranges. The dosage can be adjusted by the individual physician in the event of any contraindications. In any event, the effectiveness of treatment can be determined by monitoring the oxidative stress level by methods described above as well as those well known to those in the field. Moreover, the anti-oxidant compound can be applied in pharmaceutically acceptable carriers known in the art. The application can be oral, by injection, or topical.

The anti-oxidant compound may be administered to a patient by any suitable means, including oral, parenteral, subcutaneous, intrapulmonary, topically, and intranasal administration. Parenteral infusions include intramuscular, intravenous, intraarterial, intraperitoneal or intravitreal administration. The anti-oxidant compound may also be administered transdermally, for example in the form of a slow-release subcutaneous implant, or orally in the form of capsules, powders, or granules.

Pharmaceutically acceptable carrier preparations for parenteral administration include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, emulsions or suspensions, including saline and buffered media. Parenteral vehicles include sodium chloride solution, Ringer's dextrose, dextrose and sodium chloride, lactated Ringer's, or fixed oils. The active anti-oxidant therapeutic ingredient may be mixed with excipients that are pharmaceutically acceptable and are compatible with the active ingredient. Suitable excipients include water, saline, dextrose, and glycerol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

The form may vary depending upon the route of administration. For example, compositions for injection may be provided in the form of an ampule, each containing a unit dose amount, or in the form of a container containing multiple doses.

The anti-oxidant compounds may be formulated into therapeutic compositions as pharmaceutically acceptable salts. These salts include the acid addition salts formed with inorganic acids such as, for example, hydrochloric or phosphoric acid, or organic acids such as acetic, oxalic, or tartaric acid, and the like. Salts also include those formed from inorganic bases such as, for example, sodium, potassium, ammonium, calcium or ferric hydroxides, and organic bases such as isopropylamine, trimethylamine, histidine, procaine and the like.

Controlled delivery may be achieved by admixing the active ingredient with appropriate macromolecules, for example, polyesters, polyamino acids, polyvinyl pyrrolidone, ethylenevinylacetate, methylcellulose, carboxymethylcellulose, prolamine sulfate, or lactide/glycolide copolymers. The rate of release may be controlled by altering the concentration of the macromolecule.

As used in the claims, the −180 locus of the resistin gene refers to the −180 locus as shown in FIG. 1, at which the prevailing nucleotide is C, but at which there is an SNP in which a substantial fraction of the sequences instead have a G.

The complete disclosures of all references cited in this specification are hereby incorporated by reference. Also incorporated by reference is the entire disclosure of the following paper, which is not prior art to this application: S. R. Smith et al., “A promoter genotype and oxidative stress potentially link resistin to human insulin resistance,” Diabetes, vol. 52(7) (July), pp. 1611-8 (2003). In the event of an otherwise irreconcilable conflict, however, the present specification shall control.

We claim:

1. A method for diagnosing an insulin-resistant human patient as being likely to respond favorably to anti-oxidant therapy; said method comprising determining the patient's genotype at the −180 locus of the resistin gene, and diagnosing the patient as being likely to respond favorably to anti-oxidant therapy if the patient's genotype at that locus is G/G.

2. The method of claim 1, wherein the patient has an insulin-resistance disease selected from the group consisting of diabetes mellitus, metabolic syndrome, and cardiovascular disease.

3. The method of claim 2, additionally comprising the step of treating the patient with a therapeutically effective amount of at least one anti-oxidant compound.

4. The method of claim 3, wherein the anti-oxidant compound is selected from the group consisting of vitamin C, vitamin E, vitamin A, vitamin B1, Vitamin B6, Vitamin B12, riboflavin, selenium, niacin, folic acid, biotin, pantothenic acid, choline bitartrate, inositol, PABA, lutein, zeaxanthin, Coenzyme Q10, α-lipoic acid, and lycopene.

5. The method of claim 1, additionally comprising the step of treating the patient with a therapeutically effective amount of at least one anti-oxidant compound.

6. The method of claim 5, wherein the anti-oxidant compound is selected from the group consisting of vitamin C, vitamin E, vitamin A, vitamin B1, Vitamin B6, Vitamin B12, riboflavin, selenium, niacin, folic acid, biotin, pantothenic acid, choline bitartrate, inositol, PABA, lutein, zeaxanthin, Coenzyme Q10, α-lipoic acid, and lycopene.


SEQUENCE LISTING (11 sequences)

SEQ ID NO: 1 - DNA, 21 bp, Artificial Sequence
  Synthetic primer
ttttgtcatgtttgcatcagc

SEQ ID NO: 2 - DNA, 18 bp, Artificial sequence
  Synthetic primer
gggctcagctaaccaaat

SEQ ID NO: 3 - DNA, 20 bp, Artificial Sequence
  Synthetic primer
gaccagtctctggacatgaa

SEQ ID NO: 4 - DNA, 21 bp, Artificial sequence
  Synthetic primer
agccatcaatgagaggatcca

SEQ ID NO: 5 - DNA, 19 bp, Artificial Sequence
  Synthetic primer
tccaggccaatgctgctta

SEQ ID NO: 6 - DNA, 26 bp, Artificial sequence
  Synthetic primer
tcgccggctccctaatatttagggca

SEQ ID NO: 7 - DNA, 23 bp, Artificial sequence
  Synthetic primer
tcattctctggccaattcagagt

SEQ ID NO: 8 - DNA, 22 bp, Artificial sequence
  Synthetic primer
ggagtgtgcccaatgctatatg

SEQ ID NO: 9 - DNA, 32 bp, Artificial sequence
  Synthetic primer
tctgtggcttccaagtcttagaacctcaactg

SEQ ID NO: 10 - DNA, 13 bp, Homo sapiens
tgaagacggaggc

SEQ ID NO: 11 - DNA, 13 bp, Homo sapiens
tgaagagggaggc